(19) United States
(12) Patent Application Publication
(10) Pub. No.: US 2025/0118392 A1
(43) Pub. Date: Apr. 10, 2025

(54) ANALYSIS SYSTEM FOR BIOMARKER EXPRESSION

(71) Applicants: Canon Medical Systems Corporation, Tochigi (JP); The General Hospital Corporation, Boston, MA (US)

(72) Inventors: Satoru Komatsu, Melrose, MA (US); Hyungsoon Im, Weston, MA (US); Taehwang Son, Boston, MA (US)

(21) Appl. No.: 18/909,460

(22) Filed: Oct. 8, 2024

Related U.S. Application Data
(60) Provisional application No. 63/589,202, filed on Oct. 10, 2023.

(51) Int. Cl. G16B 45/00; G01N 21/64

(52) U.S. Cl. CPC G16B 45/00; G01N 21/6456

(57) ABSTRACT

An analysis system for biomarkers expression is provided. This system is particularly useful for viewing biomarker expression on extracellular vesicles. The system includes an analysis element, a ranging and scaling element, an adjustment element, and a selection element. There provides a display that is configured to display a data of biomarkers expression in one or more graphs.

### CROSS-REFERENCE TO RELATED APPLICATIONS

The present application claims priority to U.S. provisional application No. 63/589,202 filed 10 Oct. 2023. The disclosure of the above-listed provisional application is hereby incorporated by reference in its entirety for all purposes. Priority benefit is claimed under 35 U.S.C. § 119(e).

### BACKGROUND OF THE INVENTION

### Field of the Disclosure

The present disclosure relates to systems, software, and devices for analysis of biological data. More particularly, the disclosure exemplifies how to visualize data from images containing extracellular vesicles having multiple biomarkers.

### Description of the Related Art

Extracellular vesicles (EVs) in bodily fluids have been considered promising materials for liquid biopsy by virtue of the EVs containing many kinds of proteins, miRNAs, and mRNAs on its surface or inside the vesicle. EVs are classified into several subtypes, which include exosomes, microvesicles, microparticles, ectosomes, oncosomes, apoptotic bodies, and so on, and their size has large variety with a range of around 50 nm to 5 μm.

Virtually all types of cancer cells are capable of generating EVs. Many of these EVs promote cancer progression and can influence the behavior of cancer cells. Since the concentration and contents of the EVs released by cancer cells will vary based on the cell of origin, development stage, and responsiveness to treatment, the EVs from cancer cells are an important source of diagnostic and prognostic information. (Chang W H, et al., “Extracellular Vesicles and Their Roles in Cancer Progression. Methods Mol Biol.” 2021; 2174:143-170.)

Liquid biopsies, a non-invasive alternative to tissue biopsies, can be used to analyze EVs found in circulation. When looking at the EVs from cancer cells, this can provide for diagnosis and prognosis of the cancer. Liquid biopsy on small EVs below 200 nm have been actively studied, but most of the research uses bulk assay for analyzing EVs properties because EVs cannot be observed with an ordinary optical microscope due its small size. Fluorescence microscopy is one way to make single EVs observable, but for small EVs, the fluorescence intensity is weak, and observation becomes difficult. For such small EVs and/or for samples having small EV concentration, plasmon enhancement can be used.

With fluorescence microscopy, multiple fluorescent markers may be combined in the analysis to elucidate information about different components of the EV. Single EV analysis with multiple fluorescent colors is important, but to understand the results, the number of detections and co-localization analysis of markers expressed in EVs are needed. This can increase the complexity of the display and/or analysis. For single EV analysis using imaging data, a graphical user interface can make the analysis easier and more efficient.

One method that focuses on measuring extracellular vesicle and virus size, aggregation, and biomarker colocalization (see, for example, U.S. Pat. No. 11,262,359). Another provides a simple log scale heatmap for co-localization analysis (Martel, et al., “Extracellular Vesicle Antibody Microarray for Multiplexed Inner and Outer Protein Analysis” (ACS Sens. 2022, 7, 3817-3828). U.S. Pat. No. 10,755,406 describes methods for co-expression analysis of markers in a tissue sample using a heat map of marker expression for a plurality of single marker channel images. Co-localized regions of interest are determined from the overlay masks and mapped. (Rizk et al., An ImageJ/Fiji Plugin for Segmenting and Quantifying Sub-Cellular Structures in Fluorescence Microscopy Image” Seminar Inst. Res. Biomed, (2013), pp. 1-26.

However, there is still need for a simple and intuitive interface to provide the information to doctors, clinicians, researchers, or whoever needs the data. Thus, there is provided an analysis system, apparatus, and computer-implemented methods for the analysis of biomarkers.

### SUMMARY OF EXEMPLARY EMBODIMENTS

According to at least one embodiment of the disclosure, there is provided an analysis system, apparatus, and computer-implemented methods for the analysis of biomarkers. The system, apparatus and method comprises: an input, the input comprising: one or more fluorescence images, each image being an image of a substrate that was treated with two or more biomarkers, and biomarker standard expression profiles for each of the two or more biomarkers; a processor comprising: an analysis element that obtains data of biomarker expression from the one or more fluorescence images, a ranging and scaling element configured to adjust a range and scale according to data of biomarker expression or biomarkers standard expression profile, an adjustment element configured to adjust a threshold of the data of biomarker expression, wherein a single variable, α, is used to adjust the threshold for each of the two or more biomarkers; a selection element configured to select displayed biomarkers and/or displayed combination of co-localization. The system, apparatus and method further comprises: a display configured to display a data of biomarkers expression in one or more graphs.

These and other objects, features, and advantages of the present disclosure will become apparent upon reading the following detailed description of exemplary embodiments of the present disclosure, when taken in conjunction with the appended drawings, and provided claims.

### BRIEF DESCRIPTION OF DRAWINGS

The patent or application file contains at least one drawing executed in color. Copies of this patent or patent application publication with color drawing(s) will be provided by the Office upon request and payment of the necessary fee.

Further objects, features and advantages of the present disclosure will become apparent from the following detailed description when taken in conjunction with the accompanying figures showing illustrative embodiments of the present disclosure.

FIG. 1 is a system diagram of an exemplary embodiment.

FIG. 2(a) and FIG. 2(c) provide exemplary graphs showing concentrations of various biomarkers. FIG. 2(b) and FIG. 2(d) show a representations of fluorescence images with bounding boxes for biomarkers. FIG. 2(c) and FIG. 2(d) represent the changes seen in the graph of FIG. 2(a) and image of FIG. 2(b) when the threshold is changed.

FIG. 3(a) and FIG. 3(c) provide exemplary graphs showing concentrations of one or more biomarkers. FIG. 3(b) and FIG. 3(d) show a representations of fluorescence images with bounding boxes for biomarkers. FIG. 3(c) and FIG. 3(d) represent the changes seen in the graph of FIG. 3(a) and image of FIG. 3(b) when a single biomarker is selected. Only selected channels images are overlayed and displayed.

FIG. 4(a) and FIG. 4(c) provide exemplary graphs showing concentrations of various biomarkers and biomarker co-localization and contain a listing of markers. FIG. 4(b) and FIG. 4(d) show a representations of fluorescence images with bounding boxes for biomarkers. FIG. 4(c) and FIG. 4(d) represent the changes seen in the graph of FIG. 4(a) and image of FIG. 4(b) when the display is switched between biomarker detection and co-localization.

FIG. 5(a) provides an exemplary graph showing concentrations of various biomarkers where the co-localization reference is selectable. FIG. 5(b) is another graph showing the co-localization of biomarkers as a Venn diagram. FIG. 5(c) is another Venn diagram showing co-localization of biomarkers. FIG. 5(d) is another graph showing the co-localization of biomarkers as a 2D scatter plot. FIG. 5(e) is another graph showing the co-localization of biomarkers as a 3D scatter plot. The co-localization reference is selectable.

FIG. 6(a) and FIG. 6(c) provide exemplary graphs showing concentrations of various biomarkers where the subtype and drug may be selected. FIG. 6(b) and FIG. 6(d) show a representations of fluorescence images with bounding boxes for biomarkers. FIG. 6(c) and FIG. 6(d) represent the changes seen in the graph of FIG. 6(a) and image of FIG. 6(b) when the subtype and/or drug is changed. By selecting cancer subtype and/or drug, only related markers are shown in the GUI.

FIG. 7(a) provides an exemplary graph showing concentrations of various biomarkers. FIG. 7(b) shows a representations of fluorescence images with bounding boxes for biomarkers. FIG. 7(c) is graph showing marker concentrations (yellow and/or diamond) and a color-coded or pattern-coded range for standard and cancerous tissue.

FIG. 8 is a flowchart showing the workflow according to some embodiments as described herein.

FIG. 9 is a flowchart showing the workflow according to some embodiments as described herein.

FIG. 10 is a diagram overview for particle detection.

FIG. 11 is a flowchart showing particle detection.

FIG. 12(a) is an exemplary section of an image and FIG. 12(b) is a flowchart showing the formation of bounding boxes 1.

FIG. 13(a) is an exemplary section of an image and FIG. 13(b) is a flowchart showing the formation of bounding boxes 2.

FIG. 14 is a flowchart showing co-localization with an overlap method as shown in FIGS. 5(c) and 5(d).

FIG. 15(a) is an exemplary section of an image showing a map of the reference channel. FIG. 15(b) is an exemplary section of an image of a different channel from FIG. 15(a). FIG. 15(c) is a flowchart showing co-localization 2, a threshold method.

Throughout the figures, the same reference numerals and characters, unless otherwise stated, are used to denote like features, elements, components or portions of the illustrated embodiments. Moreover, while the subject disclosure will now be described in detail with reference to the figures, it is done so in connection with the illustrative exemplary embodiments. It is intended that changes and modifications can be made to the described exemplary embodiments without departing from the true scope and spirit of the subject disclosure as defined by the appended claims.

### DETAILED DESCRIPTION OF EXEMPLARY EMBODIMENTS

An imaging system has been developed for diagnosis, characterization, prognosis, or treatment progression. The system is particularly useful when the sample on a substrate has a low concentration of components for analysis, such as EVs labeled with biomarkers.

EVs cannot be resolved with an optical microscope due to their small size. In order to detect them with a microscope, a fluorescent dye is attached and its fluorescent light is detected as a bright spot to detect the presence or absence of the fluorescence signal. Because of the small size, there are fewer fluorescent dyes to attach to, and the fluorescent signal is weak, so amplification of the signal is required. One useful method is to use plasmon resonance. However, the signal may still be low, and with different fluorophores with different quantum efficiencies, binding capacities, and enhancement characteristics, there is a broad range of detection.

For immunofluorescence staining of EVs, a substrate (such as a plasmon enhancing substrate) is functionalized so the surface would capture the EVs by covalent bonding. Labeled EVs are loaded onto the substrate, fixed, and blocked. The EVs are then fluorescently labeled by sequential incubation with primary and secondary antibodies. With the 4-channel imaging and QUAD markers, four different signals can be seen in the fluorescence image. Each EV may be labeled with 1, 2, 3, or all 4 of the biomarkers, adding a complexity of the analysis for diagnosis, characterization, prognosis, or treatment progression that not simple to convey to the clinician or other user is appropriate detail. In some embodiments, plasmon enhanced fluorescence is used for imaging. Plasmon enhancements have been characterized in each of four channels (AF488, AF555, AF647, and Cy7). See, U.S. Pat. Pubs. 2023/0160809 and 2023/0123746, which are herein incorporated by reference in their entirety.

An assay for the molecular profiling of tumor-derived EVs using QUAD markers (EpCAM, EGFR, HER2, MUC1) is provided where the QUAD marker levels on tumor-derived EVS show an excellent correlation with those measured in originating cells, showing that EVs can be used as surrogate liquid biopsy markers for tumor cells. EVs labeled with these and/or other biomarkers may be used for the diagnosis, characterization, prognosis, or treatment progression of cancers, such as breast cancer.

Drug-resistant and prognostic may also be imaged. For example, biomarkers for paclitaxel (P-glycoprotein and Survivin), which showed elevated protein levels in cells with higher resistance (i.e., higher IC50 values) to paclitaxel and their EVs have also been analyzed and can be used to monitor treatment. See U.S. Pat. Pub. 2024/0240256.

### System Overview

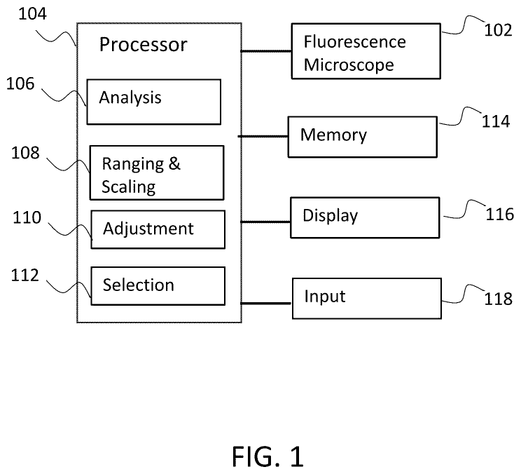

The imaging system as shown in FIG. 1 includes a fluorescent microscope 102 that is connected to and controlled by, a processor, 104. There are multiple elements within the processor, including an analysis element 106, a ranging and scaling element 108, an adjustment element 110, and a selection element 112. Each of these elements may be part of a single processing unit or may be separate. The imaging system will also include a memory 114 and a display 116. An input, such as a mouse, keypad, touchpad, is also in communication with the processor to provide input from a user. In use, a sample on a substrate is placed in the fluorescent microscope 102. The processor 104 controls total system process which are acquiring images with controlling the fluorescent microscope, analysis, ranging and scaling, adjustments, and selection. The processor 104 analyses the data form the fluorescence microscope image. The memory 114 stores the acquired images temporarily and the calculated data. The display 116 shows data of the system, such as the information of the microscope control, the live images, the acquired images, and the calculation results. This display will include the graph(s) as described herein.

A multiple channel fluorescence microscope 102 is used to image the labeled EVs bound to the substrate. The multiple biomarkers signals are then displayed in the display 115. Thus, there is provided a system and method to display graphs and/or images of the detected biomarkers that can be intuitive to understand by the researcher and/or clinician. There is also provided a system and method to modify the displayed graphs and/or images in a way that is intuitive and informative.

The images from the multiple channel fluorescence microscope 102 are sent to the processor, which will also have information on standard expression profiles for each of the biomarkers. The standard expression profile is obtained through analysis of, for example, the expression of the biomarker concentration from cancer patients as compared to patients without cancer. This may be provided as a table or database, and may be updated as more information is obtained about any biomarker.

The image analysis can also apply to particulate images and is not limited to fluorescence. An analysis of a dark field image or bright field can be accomplished as well. In some embodiments, all images are fluorescent images. In other embodiments, the images used are both fluorescent and darkfield images (e.g., the EVs are detected via darkfield, and biomarkers are detected via fluorescence.)

### Ranging and Scaling

Since each of the markers are distinct, automatic ranging and scaling for each marker expression is provided. The automatic ranging and scaling are based on standard range or reference values for each of the biomarkers. These are determined, for example, through analysis of the markers and their concentrations seen in normal and cancer cells, or for cancer cells that are receptive to a therapeutic and cancer cells that are not response to the therapeutic. In addition, clinical trials will determine the standard range of each marker expression. When displaying co-localization, it is appropriate to automatically set the expression amount of the reference marker as the maximum range.

In some embodiments, the number of detections of EV is provided on a linear scale. The percentage of co-expression between two or more biomarkers is provided on a linear scale. The total number of biomarkers is provided on a linear scale. In contrast, the estimated value converted to per mL is provided on a logarithmic scale. When displaying the number of detections of EV and markers expression or co-expression simultaneously, the numbers of biomarkers may be extremely small relative to the number of detections of EV, in which case it is better to display the number of detections of EV as logarithmic and the numbers of biomarkers as linear. In some embodiments, instead of percentages, other metrics can be provided, and may be in a linear or logarithmic format. For example, counts may be shown, both counts and percentages may be shown, or a scale providing counts relative to a specified value may be shown.

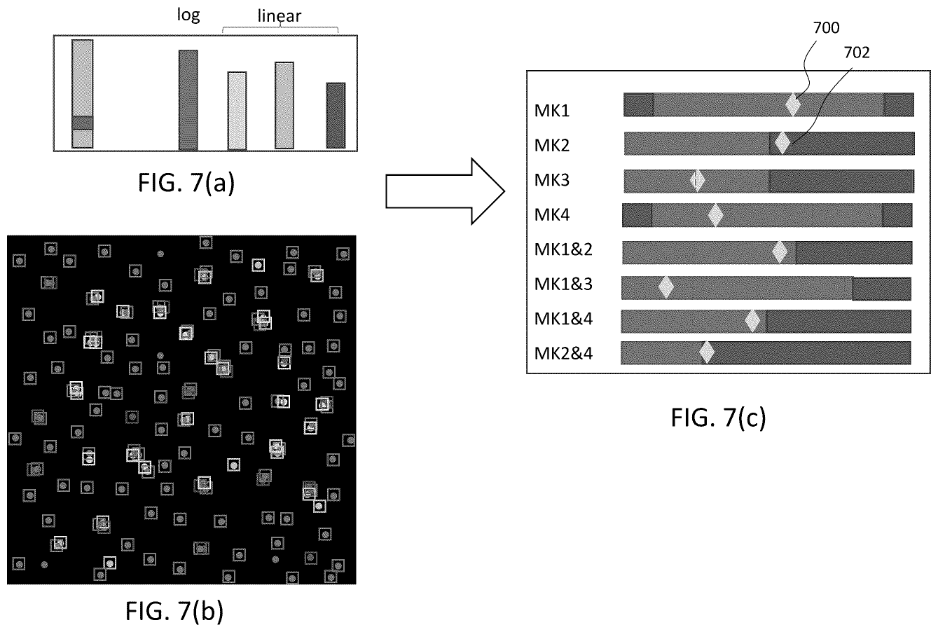

It is considered an easy-to-use and easy-to-see feature for doctors that the automatic ranging and scaling are based on standard range or reference values for each of the biomarkers. By displaying the standard range while displaying a biomarker expression of a sample against it, it is easily identifiable whether the marker expression is in the standard range or not, indicating FIG. 7(c). For bio-researchers, it is good to leave manual adjustments as well, since the study needs to look at a variety of values.

### Adjustment/Threshold

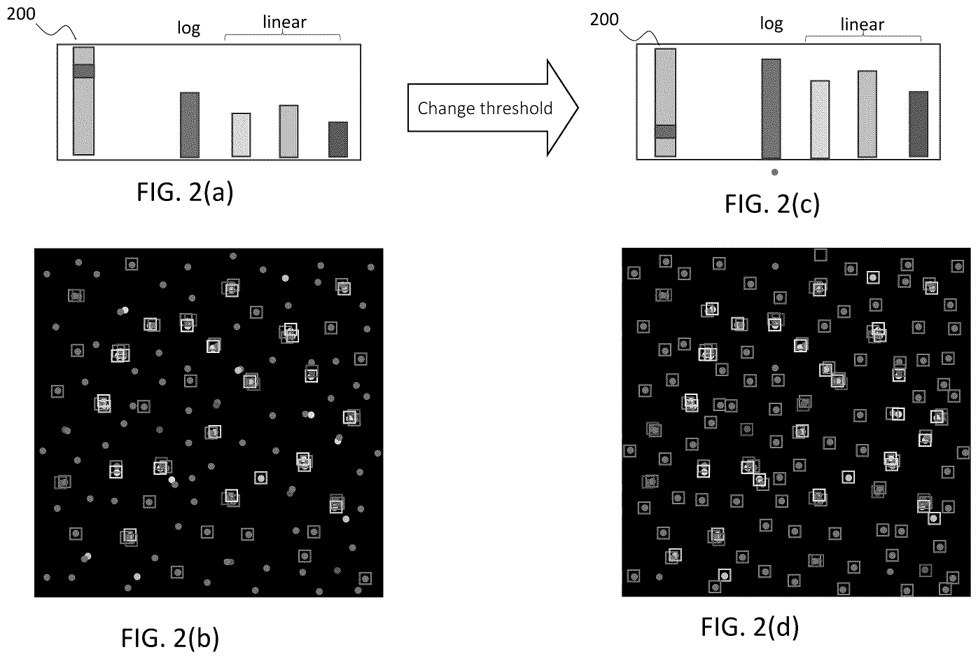

A single control unit for the thresholding is provided to all for thresholding all markers simultaneously. All markers may be adjusted or select biomarker(s) may be adjusted independently. This control unit may be manipulated, as shown in FIG. 2(a), with a slider that is moved up or down via a touchscreen or another input device (e.g., a mouse).

EV detection results vary depending on the thresholds. Although it is sometimes possible to detect it successfully with a pre-set threshold, sometimes user need to adjust them, if the number of detections is significantly abnormal case, etc. However, it is inefficient to adjust each marker channel separately. It is desirable to be able to properly adjust the thresholds of several different biomarkers by adjusting one value. For example, multiple thresholds are changed simultaneously by α by setting the variable as α and multiplying it by a value specific to each biomarker. More specifically, mean value (Mᵢ) and standard deviation (Sᵢ) could be determined for each channel image of different biomarker, and Tᵢ=Mᵢ+αSᵢ could be set as the threshold value, where i represents a different biomarker channel. It is also hard to do without proper information with display. Biomarkers' expression levels are broad, so it is difficult to see the difference on bar graphs with same range or scale, however, the user is often limited to a single display or limited viewing area for this information, so the information needs to be provided in a manner to optimize value to the user. The changed threshold facilitates user adjustment by displaying real-time updates of the changed detection results. For examples, the numbers of detections FIG. 2(a) to FIG. 2(c) or the bounding boxes of the detections results FIG. 2(b) to FIG. 2(d). When the threshold is set high as shown in FIG. 2(a), the biomarkers with weak intensity are not detected on the image as shown in FIG. 2(b). Here, just by checking the bar graphs as shown in FIG. 2(a), it is unclear if there are undetected biomarkers, but by checking the image and detection results in FIG. 2(b), it is possible to confirm the undetected biomarkers. Lowering the threshold as shown in FIG. 2(c) can prevent the undetected biomarkers, and at the same time, by checking the image as shown in FIG. 2(d), the optimal threshold can be easily set.

### Selection

The display of biomarkers and/or display of combinations of co-localization may be defined by the system, or it may be modified by a user via the selection element 112. This selection element may provide input from the user selecting one or more biomarkers, etc. from a list on the display.

While some embodiments focus on the ability to select and view one or a sub-combination of biomarkers and/or their combinations, other embodiments provide the ability to select and view a display that where the marker selection is defined according to cancer type or subtype (e.g., Luminal A or Luminal B cancers). This selection may be automatic based on a system-defined or user-defined preference.

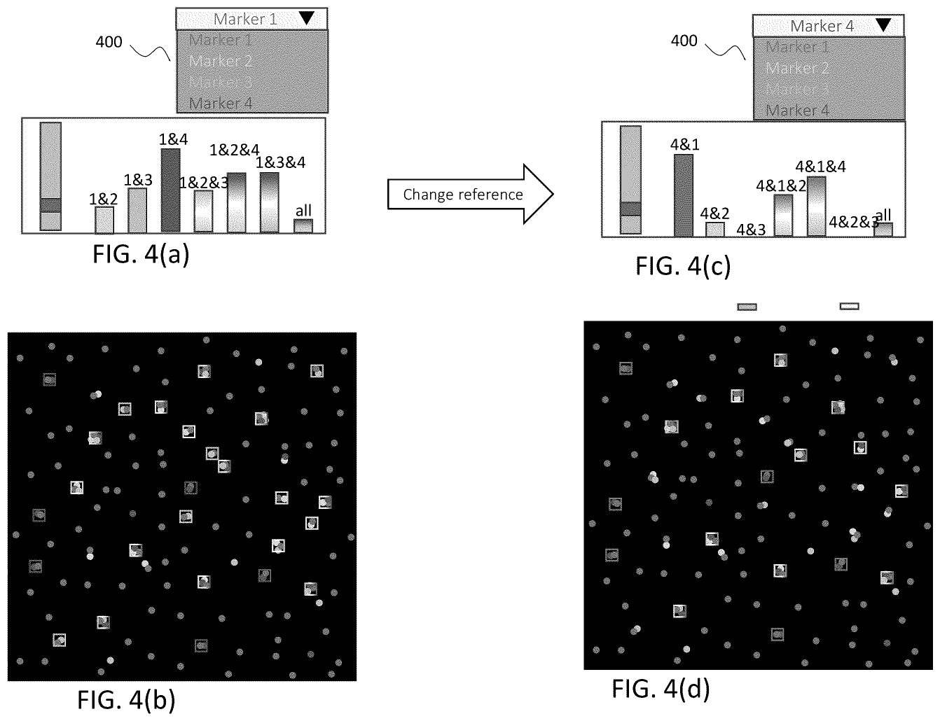

In research applications, it is useful to specify the combination of biomarkers in manual operation to ascertain the various co-localizations. For example, using a marker as a reference, it is possible to calculate EVs that have both reference marker and other biomarkers to determine which marker is important. Where the reference means that EVs with the reference marker are the population. FIG. 4 shows examples of changing the reference marker. FIG. 4(a) and (b) indicate co-localization results with marker1 as the reference and FIG. 4(c) and (d) indicate co-localization with marker4 as the reference.

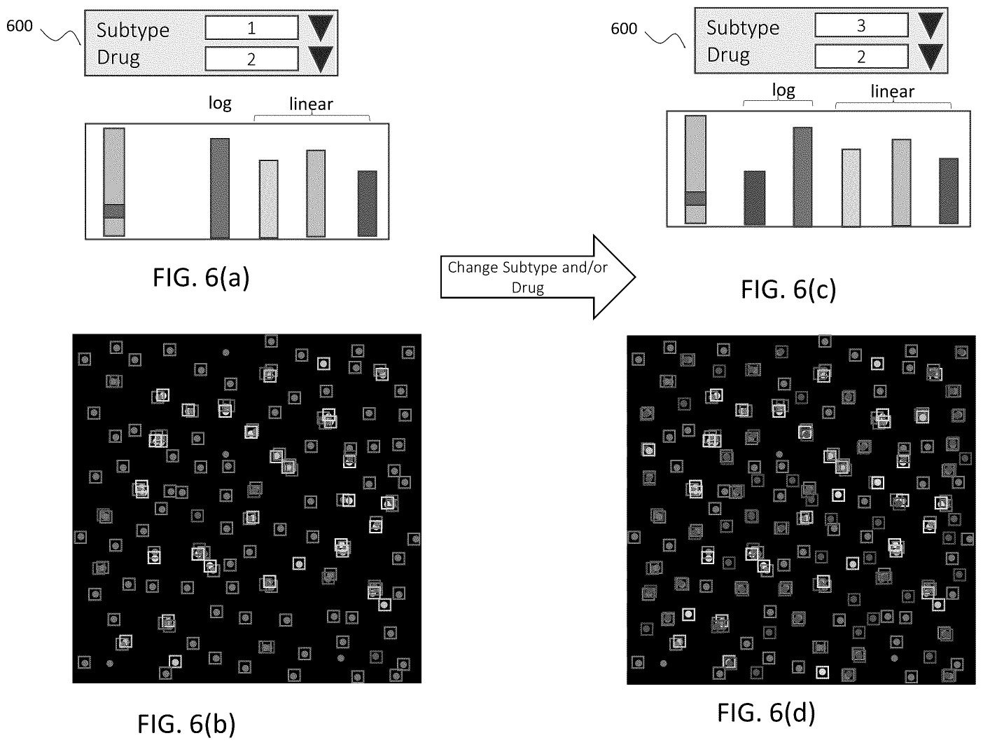

On the other hand, in clinical applications, doctors can be used to automatically select and display only the previously identified important biomarkers combinations based on cancer type, subtype, and drugs. FIG. 6 shows the examples of changing selected biomarkers and their combinations automatically based on pre-determined biomarkers and combinations.

### Exemplary EV Analysis on NPOP Substrate

Single EV analysis using plasmon-enhanced NPOP substrate is done using conventional immunolabeling of EVs with brief modifications. The NPOP substrate is described in (Adv. Funct. Mater. 2019, 1904257), provides a wide plasmonic hotspot in a 3D structure by attaching nm-sized AuNPs onto a 10 nm gold nanopillar structure. For EV capture, the substrate was fictionalized with a mixture of carboxylated and methylated polyethylene glycol (PEG) as previously described (Advanced Science 10 (8), 2205148). For plasma-derived EVs, antibodies against cancer marker were immobilized onto the NPOP substrate by physisorption method to bind the EVs have affinity to the antibodies as previously reported (ACS Appl Mater Interfaces 2022, 14, 26548-26556). Furthermore, the antibody concentration for the EV or cancer markers were further optimized to reduce the false-positive signals. Finally, the multiplexed and sensitive detection of protein markers at the single EV levels was achieved by NPOP substrate.

EVs were labeled with AF488, AF555, AF647, and Cy7 TFP dyes and immobilized onto substrates using Cell-Tak, a bioadhesive molecule, under consistent binding conditions following serial dilution of the EV samples. Analysis of the fluorescence signals from EVs attached at specific concentrations revealed significant signal amplification in the AF555, AF647, and Cy7 channels. The utilization of NPOP in single EV analysis enables the detection of a higher number of EVs that are relatively less abundant in plasma, thereby facilitating the sensitive detection of cancer cell-derived EVs.

The molecular profiling capability of NPOP substrate was evaluated for the QUAD (MUC1, HER2, EGFR, EpCAM) in breast cancer cell-derived EVs. Firstly, EVs derived from breast cancer cell lines, including SKBR3, MCF7, BT474, and MDA-MB-231 cells, were then labeled with AF555 and attached to the functionalized substrate with SH-PEG-COOH liker. Subsequently, immunofluorescence staining was conducted using optimized concentrations against respective antibodies, followed by labeling with AF647 dye. The number of AF647-labeled EVs (marker channel) showing co-localization with the AF555 signal (EV channel) was analyzed and converted into co-localization percentage values. In SKBR3EVs, markers exhibiting high co-localization were HER2 and EpCAM. In MCF7, MUC1 and EpCAM showed high co-localization, while in BT474, HER2 and EpCAM were high with moderate expression of MUC1, while in MDA-MB-231, EGFR exhibited only higher expression The high EV molecular profiling capability of NPOP substrate single EV analysis as shown.

Multiplexed single EV analysis was conducted using the NPOP substrate through multi-color imaging. Breast cancer cell-derived EVs were labeled with AF555 dye and attached to the substrate. For immunofluorescence staining, CD mix (a rabbit-originated antibody mixture of CD9, CD63, and CD81) was assigned to the AF488 channel, QUAD mix (a mouse-originated antibody mixture of MUC1, HER2, EGFR, and EpCAM) was assigned to the AF647 channel, and HER2 (a rat-originated antibody) was assigned to the DL755 channel. Although HER2 is included in the QUAD antibody, an additional channel was assigned to HER2. For this purpose, negative control samples of No EV (PBS) were prepared for the EV and CD mix channels, and EVs derived from normal cells in breast tissue (HS371T) were prepared as a negative control for the QUAD channel. In the No EV sample, no signal was observed in none of the CD mix, EV, QUAD, or HER2 channels. In HS371T, a normal cell EV sample, signals were only observed in the CD mix and EV channels. Additionally, in breast cancer cell EVs, signals were observed in the CD mix, EV, and QUAD channels, while the HER2 signal was only detected in the HER2-positive cells SKBR3 and BT474 EVs. Through analysis, it was confirmed that the multiplexing capability of NPOP substrate-based single EV analysis using multi-color imaging.

Images were analyzed using ImageJ and custom code. Background intensity was subtracted using the rolling ball method (radius=20), and the ImageJ Comdet plugin was used to detect EV locations from the AF555 channel. The intensity thresholds for AF647 and DL755 channels were defined by IgG control experiments, and the threshold value was defined by mean+3×standard deviation.

### Displays

A linked display of graphs and an image may be provided. Such linked displays are provided through the application but may be exemplified by FIG. 2(a) and FIG. 2(b). Detection results are shown in both the graph of FIG. 2(a) and in the image of FIG. 2(b). In FIG. 2(b), all markers images or selected markers images are overlayed on same image area.

The analysis system as described herein proves analysis of co-localization with a high degree of freedom. A reference marker of co-localization can be switchable between the different biomarkers. Similarly, the combination of co-localization is switchable. For example, if biomarker 1 is selected as the reference marker, the co-localizations may be between this biomarker and biomarker 2, this biomarker and biomarker 3, this biomarker and biomarker 4, as well as co-localizations of three biomarkers (biomarkers 1, 2, 3; biomarkers 1, 3, 4, and biomarkers 1, 2, 4). Analysis can be displayed in different ways. For example, the scale may provide the number of EVs detected, the number of biomarkers detected, the number of co-localizations, the detected biomarkers within a standard range, etc.

Different users may have different needs for viewing the data. For example, a lab technician or oncology researcher may need to see the images with various overlays and graphs for a full analysis of the sample. A doctor may be interested only in the outcome, such as a graph with standard ranges. Each may be provided as described herein.

FIGS. 2(a)-2(d) show an exemplary analysis of a fluorescence microscope image of bound extracellular vesicles. In these figures, all EVs that are detected on the image are shown as labeled with green biomarker. There are three additional biomarkers indicated by the colors yellow, orange, and red or by the indicated patterns. The graph of FIG. 2(a) shows the total number of EVs captured and detected on the substrate (green). This is shown in a log scale. Because the biomarkers that are useful, for example, for cancer detection, are found in significantly lower concentrations that the total bond EV concentration, the graph shows the total number of EVs having a specific biomarker (shown as yellow, orange, and red or by the indicated patterns) is in a linear scale. This allows the user to see the lower detection counts of the lower expression markers in the same graph with the total number of EVs.

The image display of FIG. 2(b) shows the multiple EVs as green dots, where yellow, red, orange and red dots (or other patterned dots) indicate the EVs that are labeled with another biomarker. The graph of FIG. 2(a) is tied to the image of FIG. 2(b), where a threshold is set, are based on background noise level of each channel, which can be calculated as a standard deviation STD (σ).

FIG. 2(a) and FIG. 2(c) also show a side bar 200. This side bar 200 allows a user to control all marker thresholds. Alternatively, in some embodiments, the biomarkers may be selectable, and thus allowing control via the side bar 200 of only the selected biomarkers. The slide bar range is coefficient of the STD, e.g. [1, 6]. The range and scale of the markers expression display of FIG. 2(a) are automatically adjusted as described below. In FIG. 2(a), the slide bar 200 is set at a higher threshold. Thus, the paired image display of FIG. 2(b) can be seen with fewer bounding boxes. The bounding box's colors and/or patterns are consistent with the color and/or pattern of the bar in FIG. 2(a). In FIG. 2(b), an overlayed image of selected channels is displayed with bounding box of detected particles.

FIG. 2(c) shows the change in the graph where the slide bar is adjusted to a lower threshold. In this case, the relative intensity increases in this figure, but in the correlated image of FIG. 2(d) the number of bounding boxes for each of the biomarkers labeled is increased to account for the increased threshold.

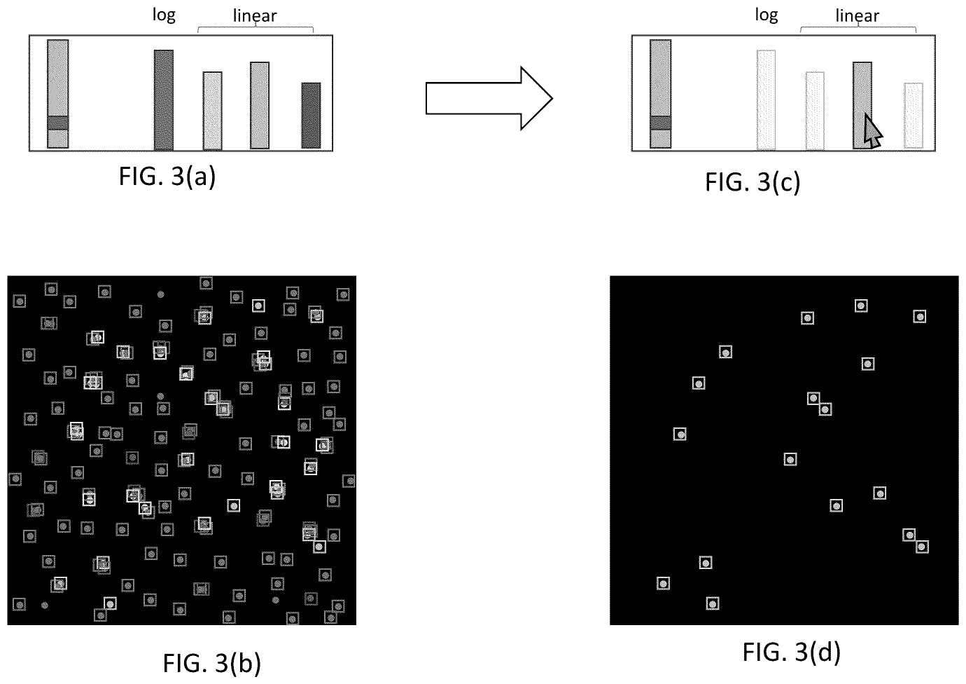

The amount of data provided with these four different biomarkers can make the data difficult to visualize. Thus, it may be the case where only selected channels images are overlayed and displayed. FIGS. 3(a) and 3(b) are the same graph and image as FIGS. 2(c) and 2(d). However, in this case, the figure shows what happens when a single channel is selected. In FIG. 3(c), the orange biomarker has been selected, making the other biomarkers turn to grey scale. The selection may be through touch screen, mouse, text, or other input device. Similarly, the deselected biomarker may be removed from the graph, or some other means of indicating either which biomarker is selected, and which biomarker is deselected may be provided. One or more biomarkers can be selected by this method. When a biomarker is selected (FIG. 3(c), the image, shown in FIG. 3(d) is transformed to show only the EVs where the selected biomarker (orange in this example) is present.

In FIGS. 4(a)-4(d), the co-localization of the various biomarkers can be visualized. The display can be switchable between detection and co-localization display (e.g., FIG. 2(a)-2(b) and FIG. 4(a)-4(b). In co-localization display, the co-localization reference is selectable. Exemplified in FIG. 4(a), the graph has a marker selection list 400, where the figure shows that marker 1 (the green biomarker) is selected.). Thus, the graph, changes, as seen in FIG. 4(c), to indicate the colocalization of the various biomarkers with the selected biomarker. The number of EVs with this colocalization is indicated by the height within the graph, where the range and scale of the biomarkers colocalization FIG. 4(a) are automatically adjusted. Also, in some embodiments, combination of co-localization can be selectable. This can be done by selecting the reference channel and some marker channels that need to be checked.

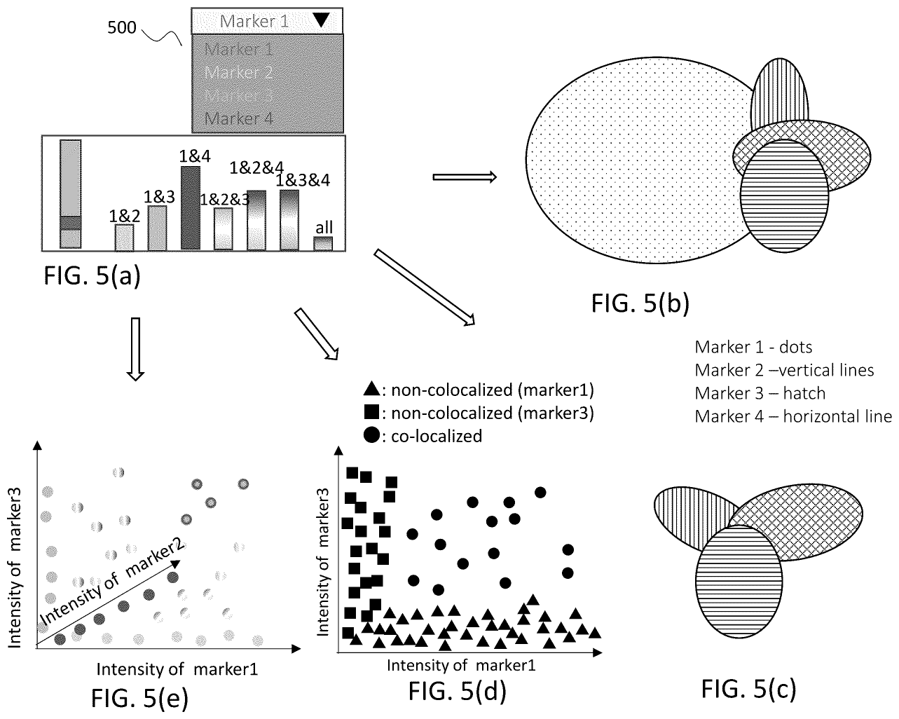

The co-localization reference can also be selectable. This is shown in FIGS. 5(a)-5(e). The reference channel can be selected from a list 500. Additionally, one or more marker channels are selected. Alternatively, the co-localizations can be shown in a different format. One such format is shown in FIG. 5(b) where Venn diagram of the overlay between the reference (green) biomarker and the Markers 2-4 is shown. In another alternative display shown in FIG. 5(c), the selected biomarkers (in this case Markers 2, 3, and 4) can be seen with their coolocalization shown as a Venn diagram. FIG. 5(d) provides the co-localization as a 2D scatter plot. This is particularly useful if only two biomarkers are selected. FIG. 5(e) provides the co-localization as a 3D scatter plot. This is particularly useful if three biomarkers are selected.

In FIGS. 6(a)-6(d), the display can inform the user about biomarker concentration and co-localization based on cancer subtype or therapeutic (drug). Other parameters that can be identified with a biomarker can also be displayed similarly. In FIGS. 6(a) and 6(c), a select box 600 allows for the selection of a specific subtype and/or specific drug. In this example, the subtype is changed from Subtype 1 (FIG. 6(a) to Subtype 3 (FIG. 6(c)). As shown in the examples in FIGS. 6(a)-6(d), a large number of biomarkers may be measured. In some of these cases, the different fluorescent channels may require separate measurements for each cancer subtype or drug. Such separate measurements may be integrated and displayed as provided in FIGS. 6(a)-6(d) and elsewhere in this document.

FIGS. 7(a)-7(c) demonstrate a display that is switchable between data analysis (raw data, FIGS. 7(a) and 7(b) and clinical (showing standard range, FIG. 7(c)) display. Each marker in FIG. 7(c) has different range based on standard detection value, shown in green, and a range outside of the standard that may be present in non-standard (e.g., cancer) populations. For example, the marker MK1 is seen in the example if FIG. 7(c) as being within the standard range 700. In contrast, the marker MK2 is outside the standard rang as shown by diamond 702. In this embodiment, the standard range can be defined by general or by a specific individual. For each of the embodiments showing displays as discussed herein, the scale may be the number of detections on the imaged substrate. Alternatively, the scale may be particles per mL. Other scales may alternatively be used.

FIG. 7(c) also provides information for the combination of biomarkers (e.g., MK1 and MK2). This provides additional information to the user, where the important feature may be the combined value. In other embodiments (not shown) the information provided may be of, MK1 or MK2. This is particularly useful when multiple biomarkers are found within a single wavelength channel when displaying the results of integrating separately measured biomarkers.

In FIG. 7(c), each biomarker display may be data from a single biomarker, for example, a biomarker that has been captured by one of the Quad markers. In other embodiments, one or more of the biomarker displayed may be from two or more biomarkers that are imaged in the same channel of a multi-channel fluorescent microscope.

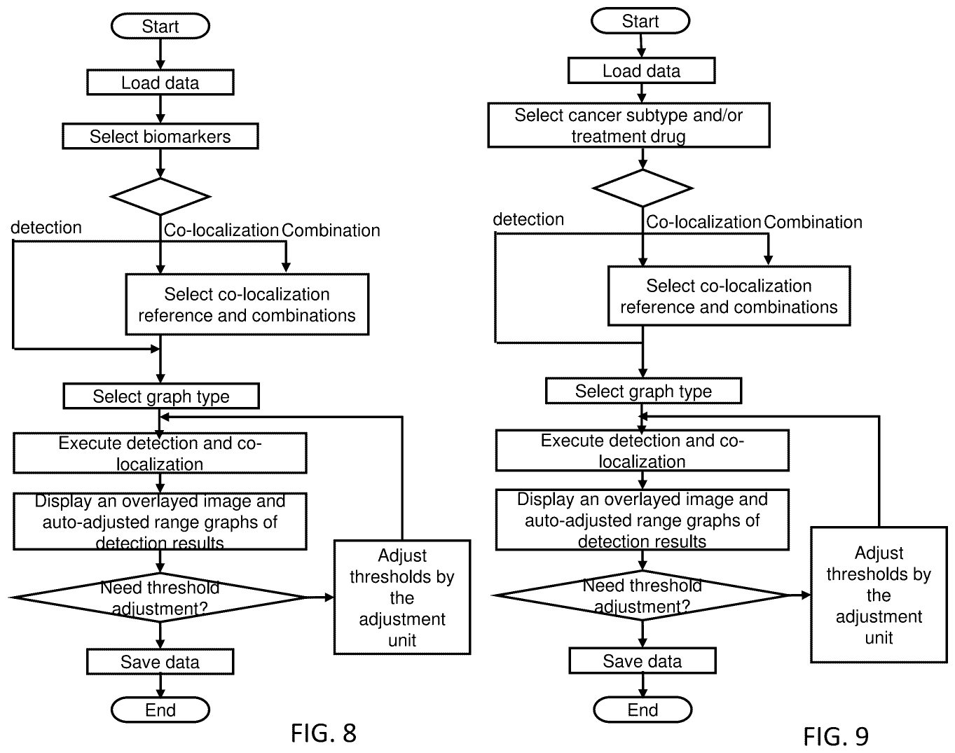

FIG. 8 provides a flow diagram for some embodiments as described herein. After starting, data is loaded into the processor/system. This data may be an image from fluorescence microscope where multiple biomarkers have been incubated on an EV sample. First, the data is loaded, then the biomarkers are selected. Then a decision is made concerning what will be displayed: detection, co-localization, or a combination thereof. For co-localization or combination, the co-localization reference and/or the combinations are selected. The graph type is then selected. This may be a pre-defined selection, but alternative graph types are preferably provided. Next, the detection and co-localization is executed. Then, the processor displays an overlayed image and auto-adjusted range graphs of the detection results. If the threshold needs adjustment, the thresholds are adjusted by the adjustment unit, and the display is re-displayed.

FIG. 9 is a flow diagram for some embodiments as described herein. Similar to FIG. 8, the flow provides for visualization where a cancer subtype and/or therapeutic (e.g., treatment drug) is selected.

### Analysis

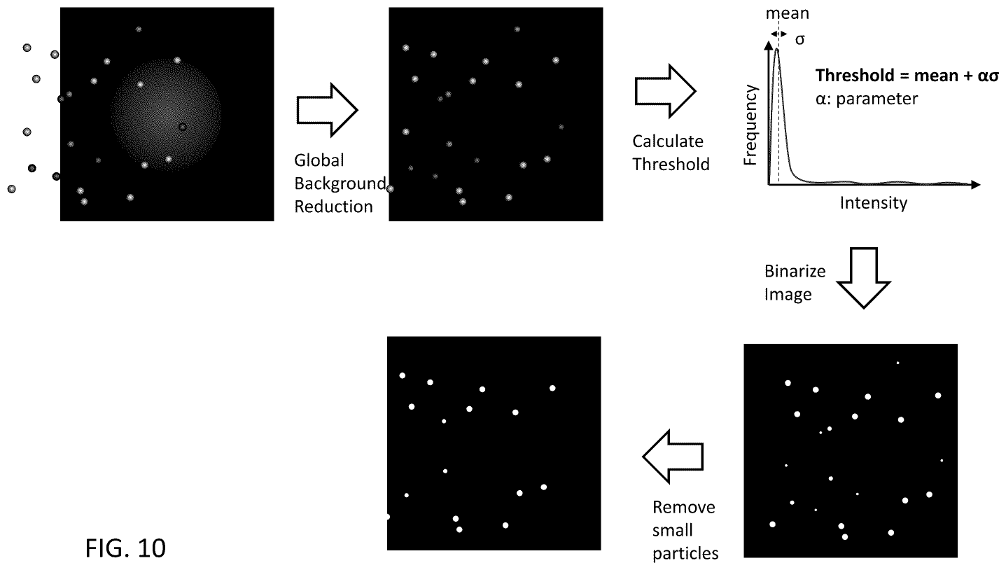

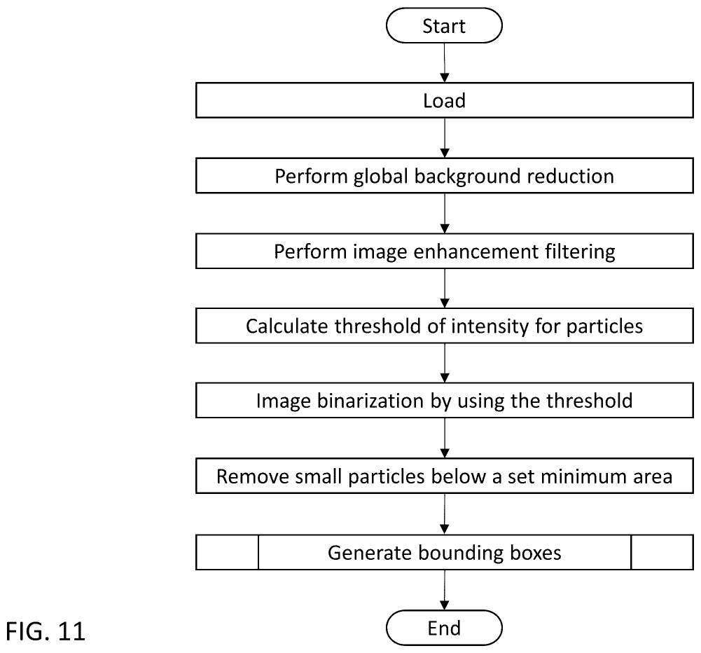

FIGS. 10 and 11 provide an exemplary method of particle detection in a fluorescence microscope image having immobilized EV particles. First, a global background reduction is performed. Then an image enhancement filtering is performed. Next, the threshold of intensity for the EV particles is calculated. In this example, the threshold is the mean+ασ, where α is a parameter and σ is the standard deviation. The parameter α can be applied equally to all the displayed biomarkers in the graph or in the image, where the mean and the standard deviation (σ) will be dependent on the specific biomarkers.

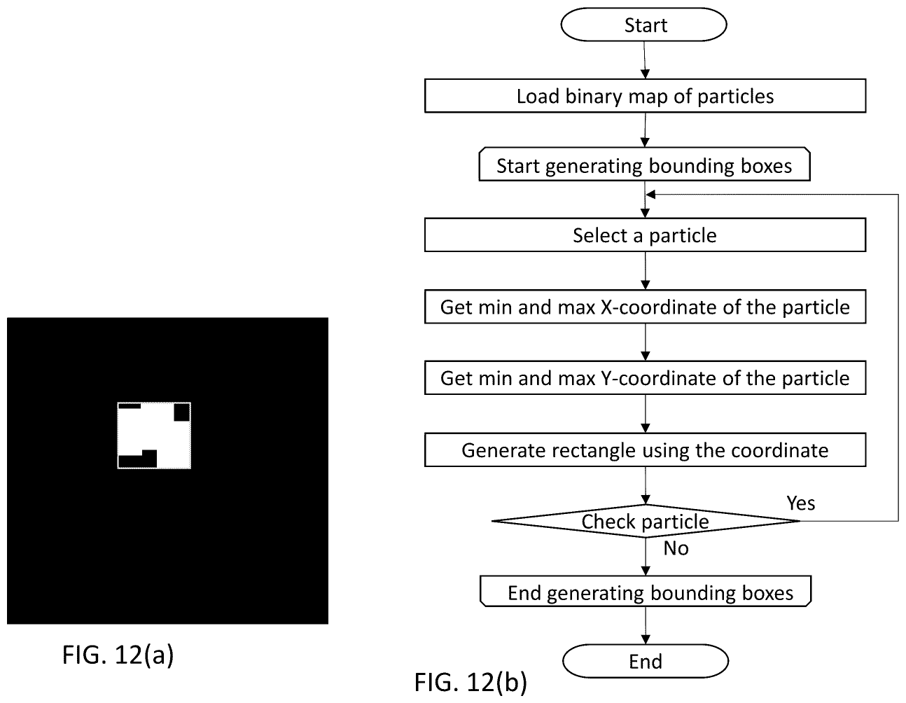

In some embodiments, bounding boxes may be created by flowing the flowchart of FIG. 12(b). A binary map of the particles is loaded, and the generation of bounding boxes is started. A particle is selected. A minimum and a maximum X-coordinate of the particle and a minimum and a maximum Y-coordinate of the particle are obtained (in any order). A rectangle is generated using the coordinates. The particle is checked, and either the bounding box generation is done, or another particle is selected. As shown in in the exemplary figure of FIG. 12(a), the shape of the detected EV after binarization is not a rectangle or a circle. From this, a rectangle bounding box is created. In other embodiments, a square may be generated (e.g., with sides the average of the X and Y coordinate) or a circle or ellipse may be generated.

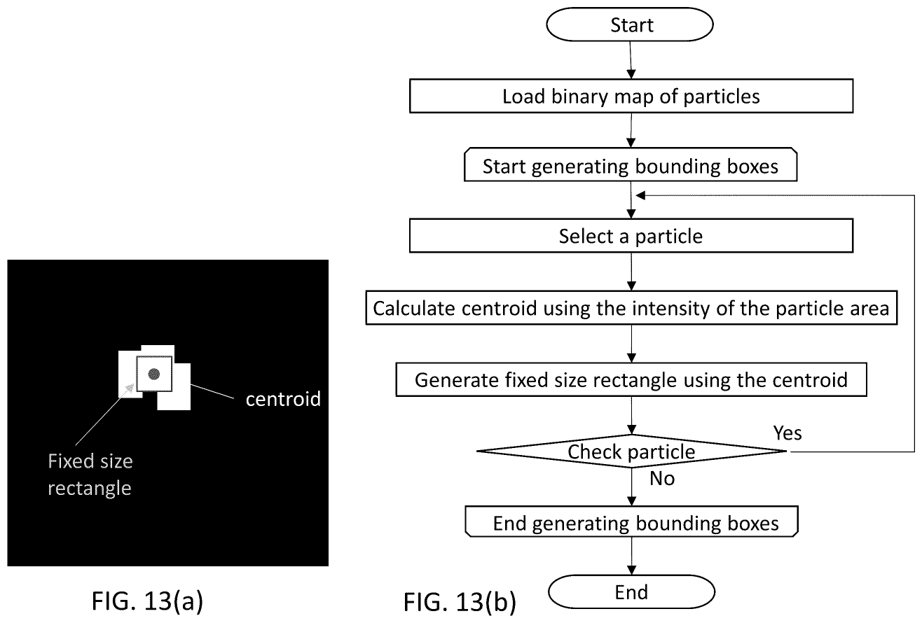

In some embodiments, bounding boxes may be created by flowing the flowchart of FIG. 13(b). A binary map of the particles is loaded, and the generation of bounding boxes is started. A particle is selected. Then, the centroid is calculated using the intensity of the particle area. A fixed size rectangle is generated using the centroid. The particle is checked, and either the bounding box generation is done, or another particle is selected. FIG. 13(a) shows that, even though the shape after binarization is not a rectangle or a circle, a predetermined rectangle (e.g., a square) is formed around the centroid as the bounding box. These embodiments provide a more uniform display of the bounding boxes.

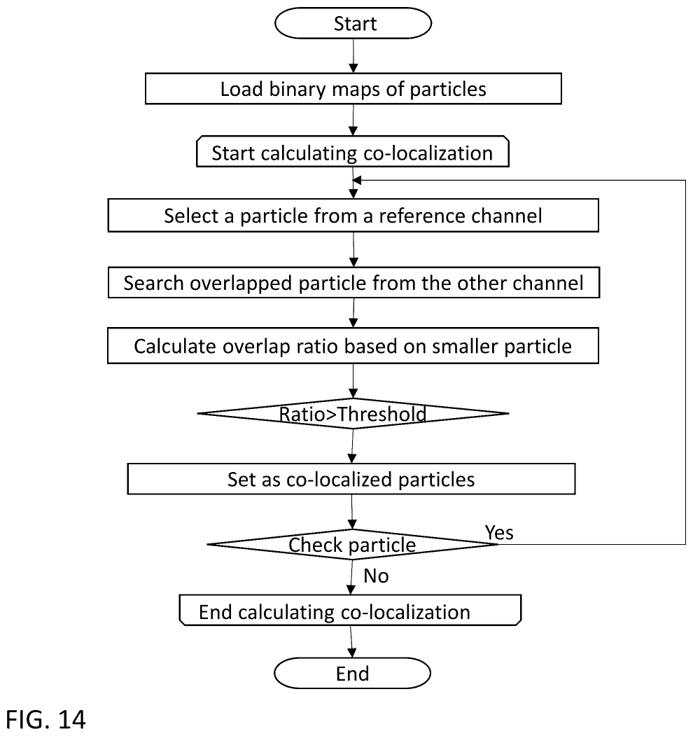

FIG. 14 provides a process for co-localization. First, a binary map of the particles is loaded into the processor and calculation of co-localization is started. A particle is selected from a reference channel and a search is performed for overlapping particles from the other channel(s). The overlap ratio is calculated based on smaller particle. If the ratio is greater than the threshold, the particles are set as co-localized particles. Then a check particle step is performed. If there are no more particles, the calculation of co-localization is ended. If yes, another particle is selected from a reference channel and the process is repeated.

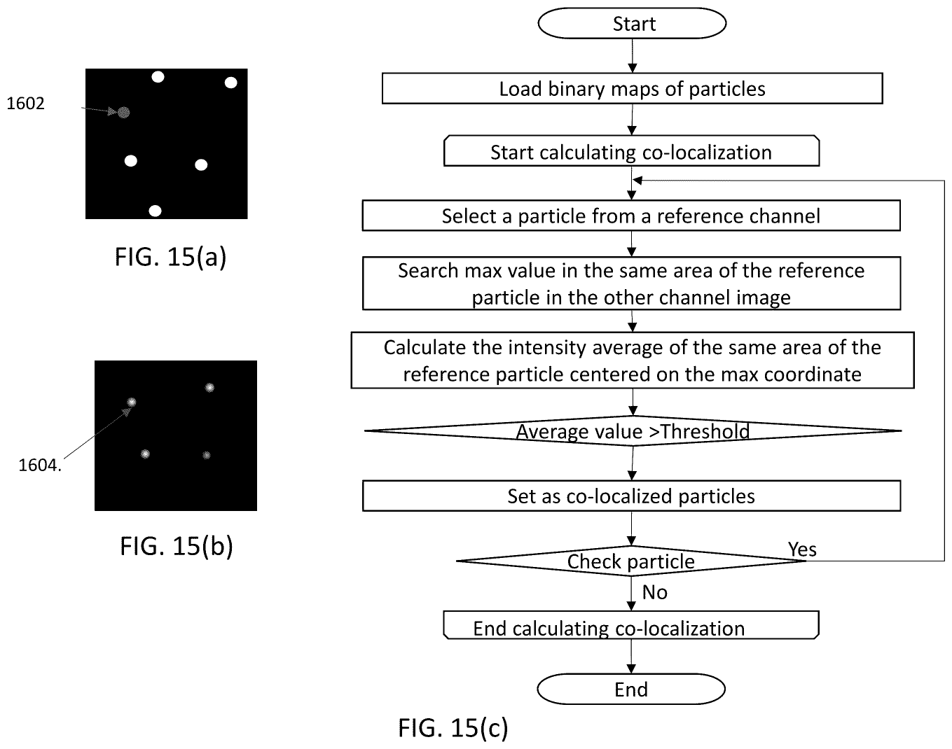

Another process of co-localization is shown in FIG. 15(c). First, a binary map of the particles is loaded into the processor and calculation of co-localization is started. A particle is selected from a reference channel and the maximum value is searched in the same area of the reference particle in the other channel image. Then, the intensity average of the same area of the reference particle centered on the maximum coordinate is calculated. If the average value is greater than a threshold value, then the particles are set as co-localized particles. Then a check particle step is performed. If there are no more particles, the calculation of co-localization is ended. If yes, another particle is selected from a reference channel and the process is repeated. FIG. 15(a) illustrates a map of the reference channel, were 1602 is the selected particle. FIG. 15(b) illustrates the image of the other channel, where 1604 is the particle in this channel where the processor will calculate the average intensity from the corresponding area.

### Definitions

In referring to the description, specific details are set forth in order to provide a thorough understanding of the examples disclosed. In other instances, well-known methods, procedures, components and circuits have not been described in detail as not to unnecessarily lengthen the present disclosure.

Unless specifically stated otherwise, as apparent from the following disclosure, it is understood that, throughout the disclosure, discussions using terms such as “processing,” “computing,” “calculating,” “determining,” “displaying,” or the like, refer to the action and processes of a computer system, or similar electronic computing device, or data processing device that manipulates and transforms data represented as physical (electronic) quantities within the computer system's registers and memories into other data similarly represented as physical quantities within the computer system memories or registers or other such information storage, transmission or display devices. Computer or electronic operations described in the specification or recited in the appended claims may generally be performed in any order, unless context dictates otherwise. Also, although various operational flow diagrams are presented in a sequence(s), it should be understood that the various operations may be performed in other orders than those which are illustrated or claimed, or operations may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “in response to”, “related to,” “based on”, or other like past-tense adjectives are generally not intended to exclude such variants, unless context dictates otherwise.

As used herein, the term “biomarker” may be used interchangeably with the term “marker” and, for use in oncology, a cancer marker. As used in relation to EV analysis, a biomarker is a molecule that is associated with an EV and can bind directly or indirectly to a capture agent or labeling agent for detecting the EV. A marker can be any components of an EV that can be recognized by a capture agent. Examples of markers include, without limitation, proteins, or nucleic acids or a component of the lipid bilayer that makes up the membrane of the EV. Useful markers include receptors (e.g., extracellular) and channel components. A marker can be either an extravesicular or an intravesicular marker, as defined herein. A marker can be present on all EVs in a sample, or on a subset of EVs in a sample. A marker that is common to all EVs in a sample is referred to herein as a pan-EV marker. The biomarker may be a surface marker. The QUAD biomarkers (EpCAM, EGFR, HER2, MUC1) are four biomarkers that have been shown to be particularly useful for diagnosis and/or characterization of cancer. The biomarkers as useful for fluorescence imaging may be labeled (either directly or indirectly) with a fluorescent moiety (e.g., a dye), such as, for example, AF488, AF555, AF647, CY3, and Cy7.

As used herein, an “extracellular vesicle” (“EV”) refers to a naturally occurring or synthetic vesicle that includes a cavity inside. The EVs comprise a lipid bilayer membrane enclosing contents of the internal cavity. An EV can include, but is not limited to, an ectosome, a microvesicle, a microparticle, an exosome, an oncosome, an apoptotic body, a liposome, a vacuole, a lysosome, a transport vesicle, a secretory vesicle, a gas vesicle, a matrix vesicle, or a multivesicular body. An EV has a dimension of up to about 10 microns, but are typically about 1000 nm or less. Exosomes and microvesicles are types of EVs, and can be shed by eukaryotic cells, or budded off of the plasma membrane, to the exterior of the cell. These membrane-bound vesicles are heterogeneous in size with diameters ranging from about 10 nm to about 5000 nm. The methods and compositions described herein are equally applicable for microvesicles of all sizes.

As used herein, the “fluorescence image” comprises a single image obtained by exiting the substrate with a single excitation wavelength or a composite image where the substrate has been sequentially excited with multiple wavelengths, and these initial images combined into one. Each initial image may show one or more types of biomarker fluorescing at the excitation wavelength.

The terminology used herein is for the purpose of describing particular embodiments only and is not intended to be limiting. As used herein, the singular forms “a”, “an”, and “the”, are intended to include the plural forms as well, unless the context clearly indicates otherwise. It should be further understood that the terms “includes” and/or “including”, when used in the present specification, specify the presence of stated features, integers, steps, operations, elements, and/or components, but do not preclude the presence or addition of one or more other features, integers, steps, operations, elements, components, and/or groups thereof not explicitly stated.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

While the present disclosure has been described with reference to exemplary embodiments, it is to be understood that the present disclosure is not limited to the disclosed exemplary embodiments. The scope of the following claims is to be accorded the broadest interpretation so as to encompass all such modifications and equivalent structures and functions.

What is claimed is:

1. An analysis system for biomarkers expression comprising:
an input, the input comprising:
one or more fluorescence images, each image being an image of a substrate that was treated with two or more biomarkers, and
a biomarker standard expression profiles for each of the two or more biomarkers;
a processor comprising:
an analysis element that obtains a data of biomarker expression from the one or more fluorescence images,
a ranging and scaling element configured to adjust a range and scale according to the data of biomarker expression or the biomarkers standard expression profile,
an adjustment element configured to adjust a threshold of the data of biomarker expression, wherein a single variable, α, is used to adjust the threshold for each of the two or more biomarkers;
a selection element configured to select displayed biomarkers and/or displayed combination of co-localization; and
a display configured to display the data of biomarkers expression in one or more graphs.

2. The analysis system of claim 1, wherein each image is an image of a substrate that has been loaded with extracellular vesicles.

3. The analysis system of claim 2, wherein the extracellular vesicles have been incubated with three or more biomarkers.

4. The analysis system of claim 3, wherein the graph provides data of EV detection on a log scale and data of individual biomarker detection on a linear scale.

5. The analysis system of claim 1, wherein the selection element is configured to obtain information from a user for the selection of biomarkers and/or combination of co-localizations to be displayed.

6. The analysis system of claim 1, wherein the selection element automatically displays the related biomarkers and/or co-localization by selecting a cancer subtype and/or a treatment drug.

7. The analysis system of claim 1, wherein the processor further comprises a display switching element configured to change display items according to the attributes from a user.

8. The analysis system of claim 1, wherein the display further comprises one or more images.

9. The analysis system of claim 1, wherein the images and the graphs in the display are changed in response to changes in the adjustment element.

10. The analysis system of claim 1, wherein the the input comprises three or more fluorescence images obtained from a multiple channel fluorescence microscope.

11. The analysis system of claim 1, wherein the input further comprising one or more dark field image of the substrate that was treated with two or more biomarkers.

12. An analysis system for biomarkers expression comprising:
an input, the input comprising:
one or more fluorescence images, each image being an image of a substrate that was treated with two or more biomarkers, and
biomarker standard expression profiles for each of the two or more biomarkers;
a processor comprising:
an analysis element that obtains a data of biomarker expression from the one or more fluorescence images,
a ranging and scaling element configured to adjust the graphs range and scale according to detected biomarkers expression or biomarkers standard expression in general or individual;
an adjustment element configured to adjust a threshold of the data of biomarker expression, wherein a single variable, α, is used to adjust the threshold for each of the two or more biomarkers, wherein the adjustment of the threshold adjusts the detection count on the graph and adjusts the display of a bonding box or boxes on the image; and
a selection element configured to select displayed biomarkers and/or displayed combination of co-localization, wherein the selection of the biomarker(s) alters the indication of biomarker(s) on the graph and alter the biomarker bounding box on the image, and
a display configured to display acquired the data of biomarker expression in one or more graphs and in one or more images.

13. A computer-implemented method comprising,
receiving one or more fluorescence images and a biomarker standard expression;
analysing the one or more fluorescence image to obtain a data of biomarker expression;
ranging and/or scaling the data of biomarker expression according to the data of biomarker expression or biomarkers standard expression profile;
adjusting a threshold of the data of biomarker expression, wherein a single variable, α, is used to adjust a biomarker threshold;
selecting one or more biomarkers to display and/or selecting one or more combination of co-localization to display; and
displaying the data of biomarkers expression in one or more graphs.

14. The computer-implemented method of claim 13, wherein each fluorescence image is a fluorescence image of a substrate that has been loaded with extracellular vesicles.

15. The computer-implemented method of claim 13, wherein the extracellular vesicles have been incubated with three or more biomarkers.

16. The computer-implemented method of claim 15, wherein displaying the data of biomarker expression comprises displaying a data of EV detection on a log scale and a data of individual biomarker detection on a linear scale.

17. The computer-implemented method of claim 13, wherein selecting one or more biomarkers and/or selecting one or more combination of co-localization comprises obtaining an input from a user input.

18. The computer-implemented method of claim 17, wherein the user input is input from user interaction with a display of a selection of cancer subtypes and/or of a selection of treatment drugs.

19. The computer-implemented method of claim 13, displaying the data of biomarkers expression in one or more graphs further comprises changing the display in response to changes in the adjustment element.
